(19) United States
(12) Patent Application Publication
(10) Pub. No.: US 2005/0233475 A1
(43) Pub. Date: Oct. 20, 2005

(54) Method of immobilizing a substrate of interest to a solid phase in a process using non aqueous or aprotic solvents

(76) Inventors: Qing Wang, New York, NY (US); Daniel M. Levine, Briarcliff Manor, NY (US); Thomas S. Parker, Brooklyn, NY (US)

Correspondence Address: FULBRIGHT & JAWORSKI, LLP, 666 FIFTH AVE, NEW YORK, NY, 10103-3198, US

(21) Appl. No.: 11/126,527

(22) Filed: May 10, 2005

Related U.S. Application Data
(63) Continuation-in-part of application No. PCT/US04/39434, filed on Nov. 23, 2004.
Application No. PCT/US04/39434 is a continuation-in-part of application No. 10/723,923, filed on Nov. 25, 2003.

(51) Int. Cl. G01N 33/553

(52) U.S. Cl. 436/526

(57) ABSTRACT

Methods are described for attaching molecules to a solid phase, such as a magnetic bead. Reactive groups on the solid surface react with a molecule that contains a sulfhydryl group. The sulfhydryl group may, but need not be protected. The molecule is then reacted with a conjugate and ligand. The linker has a reactive group, such as a maleimido group, which reacts with sulfhydryl, and also contains a ligand, such as an antibiotic, antibody, aptamer, or other molecule of interest. The invention also involves methods for quantifying usable reactive groups on a solid phase, where the sulfhydryl group containing moiety is bound, and measured. The first step of the reaction, and preferably the second step as well, take place in non-aqueous, aprotic solvent.

### RELATED APPLICATIONS

This is a continuation-in-part of application PCT/US04/39434, filed on Nov. 23, 2004, designating the United States, which is itself a continuation in part of Ser. No. 10/723,923, filed Nov. 25, 2003 and both applications are incorporated by reference in their entirety.

### FIELD OF THE INVENTION

This invention relates to methods for preparing solid phases having desired molecules, such as ligands, attached thereto. Further, the invention relates to the use of linkers to protect the activated functionality on a solid phase, and enables the determination of the number of reactive groups useable for attaching ligands to the solid phase as opposed to the total number present. The inventive processes use non-aqueous or aprotic solvents to carry out the desired steps.

### BACKGROUND AND PRIOR ART

The use of solid phase materials in the field of analytical chemistry is well known. Many methods for identifying, separating, or otherwise working with desired molecules rely on the use of solid phase materials to which a binding partner, or reactive partner, of a given molecule, is attached. Representative of the type of materials which can be used as solid phases are test tube or cuvette walls, glass slides, synthetic surfaces like plastics, particles, especially inert particles, and beads, such as magnetic beads. The latter are especially useful because they can be removed very easily from a solution in which they are placed.

The “ligand” that is attached to the solid phase, or reactive molecule, may be any substance that interacts with a target to react with it, to remove it from solution, etc. Various biological and biochemical molecules, including proteins, antibodies, carbohydrates, nucleic acids, and lipids may be the ligand, as may organic molecules, such as hormones, vitamins, antibiotics, aptamers, signalling molecules, drugs of interest, such as immuno-suppressive drugs, or any other material of interest may serve as the ligand or as a reactive material.

Due to their widespread use, it is of interest to optimize the preparation and production of solid phase materials, such as those described above. The resulting materials can be used, e.g., to determine analytes of interest when the ligand is, e.g., an aptamer, a signalling molecule, an antibody or an antigen when the target molecule is an antibody or aptamer. Anytime a binding reaction of any type is of interest, one or more components of the binding reaction may be immobilized on the solid phase.

When using the solid phases described supra, one either purchases, or places, reactive groups on their surface. These groups can be, but are not limited to amine functionalities. Other reactive groups which are useful and have been attached to solid phases as reactive moieties are epoxy, tosyl, and carboxyl groups. Others will be known to the skilled artisan.

When using these solid phase materials, it is essential for the user to know how many usable reactive groups are available for reaction with substances of interest. This is not the same as knowing how many reactive groups are on the actual surface. The manufacturer's specification of reactive groups or the number determined by chemical means, such as the ninhydrin reaction of amines, are referred to herein as the “Nominal” amount. Not all reactive groups on a solid phase are in fact available for reaction for many reasons. For example, steric hindrance is a very important consideration when reactive groups are very close to each other—as is frequently the case with solid phase materials. The proximity renders the majority of these moieties unavailable for use when linking macromolecules or even most small biomolecules. Herein, we refer to the number of reactive groups that can be used for linking a given ligand as the “Usable” amount.

Methodologies for evaluating the total number of reactive moieties on a solid phase, such as a magnetic bead, which provide the total number of reactive groups on a bead are available, but they do not measure the number of usable reactive groups.

An example of a test, for determining the nominal amount of reactive amines, is the use of ninhydrin reagent in the so-called Kaiser test. See, e.g., Kaiser et al., Biochemistry 34: 595 (1970). The test uses phenol in ethanol solution, KCN in pyridine, and ninhydrin: a ninhydrin molecule is added to the materials, where it reacts with a free amine group on the solid phase. In the reaction, the amine group is cleaved from the solid phase. The aminated ninhydrin reacts with a second molecule of ninhydrin, forming a dark blue color (Ruehmamn's blue). The intensity of the color indicates the quantity of amino groups present.

The problems with this test, which is admittedly sensitive and accurate, are manifold. First, the KCN used is toxic, and pyridine is noxious. Second, the reaction must be carried out at very high temperatures, i.e., about 120° C. Also, the test does not function when the free amino group is a part of certain molecules, such as serine, asparagine, aspartic acid, and proline. And as was alluded to, supra, the test is destructive in that it removes the free amine group. While it provides a comprehensive measurement of all amine groups, it does not give one any idea of the number of groups sterically available for reaction with a given linker.

Hence, it would be useful to have available a methodology which did not involve toxic and noxious chemicals, could be carried out at room temperature, did not require special apparatus, and which would give the number of reactive moieties on a solid surface that are useable for a given ligand.

Processes for attaching molecules to solid phases are undoubtedly known; however, as compared to the prior art, the invention described herein carries out the steps of the process in non-aqueous, or aprotic solvents. The use of these solvents prevents hydrolysis of the reactant products, which in turn permits a higher recovery of those reactant products without the complications resulting from the use of aqueous solvents. As a result of this, intermediates of, e.g., the first step of the process can be reacted directly with the reactants of the second step, without the need for purification or the resolution of complications that result from the use of aqueous solvents.

This has now been achieved, as will be seen in the discussion and exemplification, which follow.

In the disclosure which follows, the immuno-suppressive drug, rapamycin, is attached to magnetic beads; however, it is to be understood that the invention described herein relates generally to the attachment of any molecule of interest to any solid phase of interest, using the inventive methodologies set forth herein. The molecules referred to supra are exemplary, but not exclusive as to the molecules which may be attached in accordance with the invention.

### BRIEF DESCRIPTION OF THE FIGURES

FIG. 1 shows an embodiment of the invention, where a linker with a protected, sulfhydryl group is attached to an activated solid phase, deprotected to present the sulfhydryl group, which reacts with a maleimido group to form a complex to which a ligand of interest is joined.

FIG. 2 presents features of an embodiment of the invention where a linker with an unprotected sulfhydryl group, i.e., Traut's reagent, is added to the solid phase with the activated group. In such a case, deprotection is not necessary.

FIG. 3 presents a more detailed reaction scheme, where the linker of FIG. 1 is “SATA,” described infra, to which a ligand is then coupled.

FIG. 4 presents a detailed depiction of how to quantify usable groups in one embodiment of the invention.

### SUMMARY OF THE INVENTION

In brief, the invention involves several steps. First, a key reactant and facet of the invention is a solid phase which has “active” or “reactive” groups attached thereto, available for reaction. These active solid phases can be made, using standard protocols, or be purchased. Exemplary of such solid phases are magnetic beads, as described supra, but solid phases such as plastic plates, glass slides, polystyrene rods, or other materials can also be used. The reactive group may be an amine group, a hydroxyl group, a carboxyl group, a tosyl group, an epoxy group, or any other reactive group known to the skilled artisan.

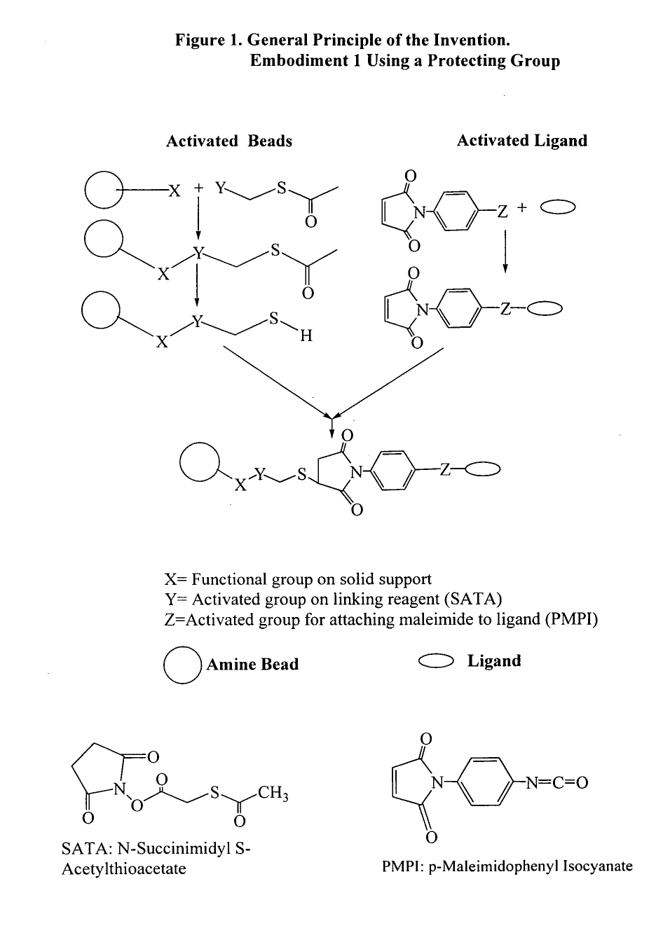

In FIG. 1, this reactive group, represented by “Y” is reacted with a linker, represented by the structure containing “Z” and the acetylated sulfhydryl group. “Z” is chosen so as to be reactive with the active group on the solid phase, and while the sulfhydryl group may be protected via acetylation, there are other protective moieties known to the skilled artisan which may be used.

The linker is permitted to react with the active group on the solid phase, to attach it thereto. Following attachment, the protecting group is removed, using techniques known to the art.

Once the sulfhydryl group is bound to the solid phase, a complex comprising a ligand and a maleimido group is added. The maleimido group reacts with the sulfhydryl group, thus binding the ligand to the solid phase. The ligand, which may be, i.e., an apatamer, a protein, an antibiotic, an antibody, or any other reactive molecule known to the art, can then be used in assays developed by the artisan.

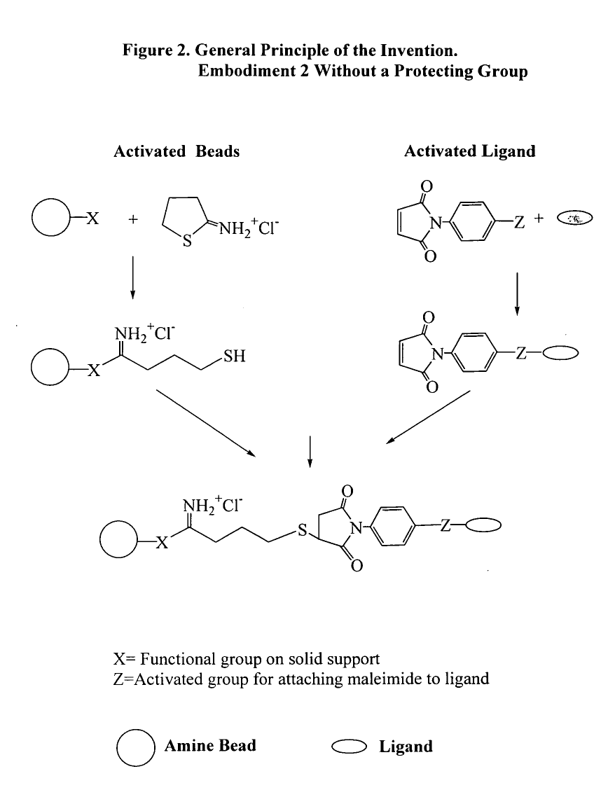

In an alternative embodiment, a free sulfhydryl group can be attached directly, i.e., without a protecting group, by using a material like Traut's reagent. This type of reaction can be desirable because there is no need to use a system where protecting groups are washed away following cleavage, i.e., it is a “one pot” system, which can be carried out at ambient, or room temperature conditions, without excesses of pH. As is seen in FIG. 2, a free sulfhydryl group is generated and is immediately useable.

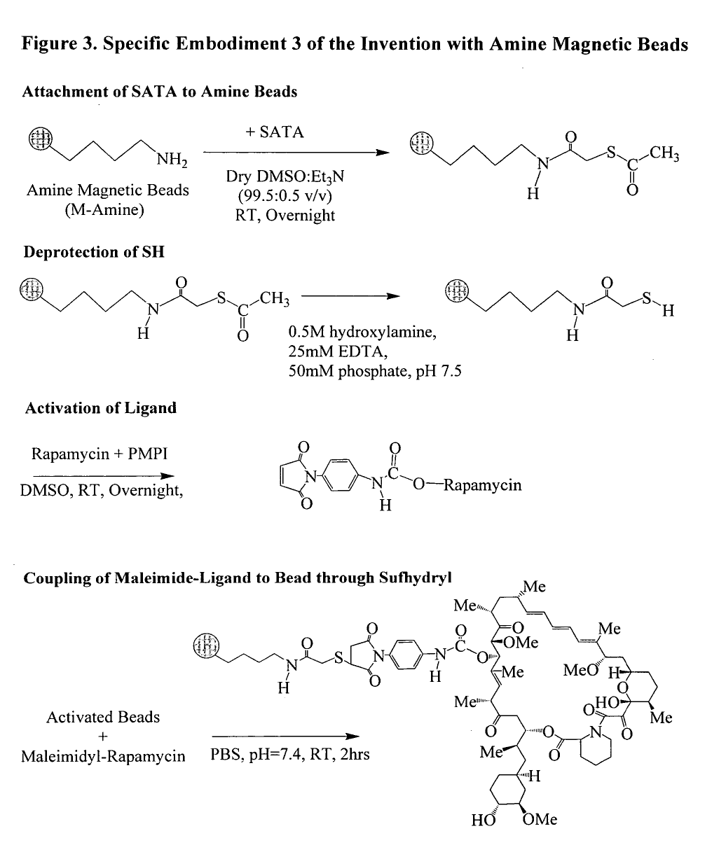

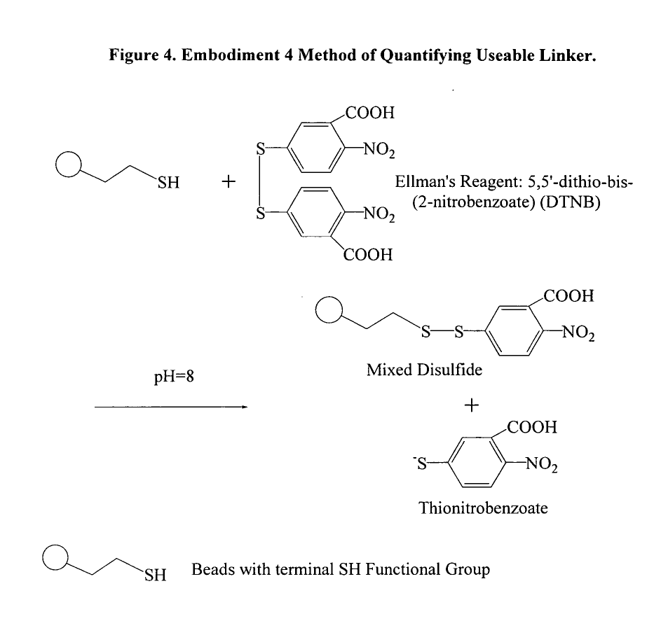

In the disclosure which follows, the specific embodiment of FIG. 3 is elaborated, as are other features of the invention, including the quantification of useable groups, such as is depicted in FIG. 4.

### DETAILED DESCRIPTION OF PREFERRED EMBODIMENTS

### EXAMPLE 1

This example illustrates the general strategy of the invention using amine beads activated with SATA and a ligand activated with PMPI. To prepare the beads used in the invention, a solution of N-succinimidyl S-acetylthioacetate (“SATA” hereafter) was prepared by admixing 2.0 mg of SATA with 0.5 ml of dimethyl formamide (amine-free DMF) containing 0.5% triethylamine. SATA is used herein because it contains both a protecting group, i.e., an acetate moiety, for a free sulfhydryl group, and an N-hydroxysuccinimide moiety, which is a good leaving group for linking to the amine bead. This solution was combined with 4×10⁹ amine group presenting beads that had been washed thoroughly. The vial containing the beads and SATA solution was covered with argon, stoppered, and then was slowly tilted and rotated at room temperature overnight.

This resulted in attachment of SATA to the beads, via an acylation reaction with the free primary amine group on the beads. The protected beads can be stored or used immediately, the free sulfhydryl group remains protected. In order to use the beads immediately, the protecting acetyl group was removed by adding a deprotecting solution (0.5 m hydroxylamine, 25 mM EDTA, 50 mM phosphate, pH 7.5). The beads were kept suspended in deprotecting solution by rotating the tube for 2 hours at room temperature, after which the hydroxylamine hydrochloride and other by-products were removed by washing with buffer (30 mM triethanolamine, 150 M sodium chloride, pH 7.3).

Following the deprotecting step, the beads with free sulfhydryl were reacted with a conjugate of a ligand and p-maleimidophenyl isocyanate. The maleimide serves as a “bridge” to link the ligand to the beads through free SATA sulfhydryl group, as seen in FIG. 1.

### EXAMPLE 2

This example parallels Example 1, but uses a linking reagent that produces a bead with an unprotected sulfhydryl group. These beads can be tested immediately to determine the number of usable linking groups or they can be mixed with activated ligand to produce the final product. Amine beads, as described supra, were activated with Traut's reagent and a ligand activated with PMPI. To prepare the beads, a solution of 2-Iminothiolane.HCl (“Traut's Reagent” hereafter) was prepared by admixing 2.4 mg of Traut's Reagent with 0.4 ml of reaction buffer (0.1 M sodium phosphate, 1 mM EDTA, pH 8.0). This solution was combined with 4×10⁸ amine group presenting beads that had been washed thoroughly. The vial containing the beads and Traut's Reagent solution was slowly tilted and rotated at room temperature for 1 hour.

This resulted in attachment of Traut's Reagent to the free primary amine group on the beads. The beads with free sulfhydryl are reacted with a conjugate of a ligand and p-maleimidophenyl isocyanate. The maleimide serves as a “bridge” to link the ligand to the beads through the sulfhydryl group.

### EXAMPLE 3

This example is a refinement of Example 1, supra with coupling of the SATA beads to maleimide-activated rapamycin.

First, sulfhydryl groups were deprotected. Magnetic beads that had protected SATA attached to them, which had been prepared in accordance with Example 1, supra, were used. The acetyl group was removed by washing 900 μl of SATA beads (1×10⁹ beads/ml) and adding 900 μl of deprotecting solution (0.5 m hydroxylamine, 25 mM EDTA, 50 mM phosphate in water, pH 7.5). The beads were then washed and combined with another 900 μl of deprotecting solution, for two hours. In this way, a hydroxylamine, rather than a reducing agent generates the sulfhydryl group. The sulfhydryl group containing beads were then washed, three times with 900 μl of PBS (pH 7.4).

Rapamycin-PMPI was then made by combining a solution of rapamycin (33.0 mg, 0.036 mmol) which had been made by dissolving 33.0 mg (0.036 mmol) of rapamycin in 0.5 ml of DMSO and then N-(p-maleimidophenyl)isocyanate, or “PMPI” was added (8.6 mg, 0.4 mmol). This solution was stirred under argon, at room temperature, protected from light, overnight. The mixture was then treated with 10 ml of diethylether, and stirred vigorously to extract DMSO. The diethyl ether was then decanted and the resulting yellow oil was triturated with methylene chloride, yielding a yellow powder. The powder was suction filtered, washed with methylene chloride, and vacuum dried, and purified via flash chromatography using methylene chloride/methanol (99:1) as eluent.

This material was then coupled to the beads. A total of 1.2 mg of the rapamycin-PMPI dissolved in N,N′-dimethyl formamide was added to the beads so that the final concentration of N,N-dimethyl formamide was less than 10% v/v. Following incubation at room temperature for 2 hours, the tubes were contacted with a magnet to remove the beads from supernatant. The beads were washed, three times, with the PBS buffer referred to supra, and then 900 μl of the PBS buffer was added to make a solution of 1×10⁹ beads/ml.

### EXAMPLE 4

This example of the invention shows the use of Ellman's reagent to measure the number of usable SH groups on the beads. More details are given in FIG. 4.

First, SATA containing beads were deprotected, so they could react with Ellman's reagent. A small sample of SATA beads from Example 1 supra (75 ul, 1×10⁹ beads/ml) were treated with deprotecting solution supra (0.5 m hydroxylamine, 25 mM EDTA, 50 mM phosphate, pH 7.5) for 2 hours at room temperature.

The resulting beads were suspended in 100 ul of the reaction buffer described supra (0.1 M sodium phosphate, 1 mM EDTA, pH 8.0). Ellman's reagent (DTNB: 5,5′-dithio-bis(2-nitrobenzoate)) was added (5 ul, 4 g/ml). Ellman's reagent reacts with free thiol groups, such as those on the unprotected SATA molecules, and releases colored thionitrobenzoate, which was measured at 412 nm.

A nominal range of 0.1-0.2 mmole/g of total amine was provided for on the beads used herein. When these beads were tested using the ninhydrin test, the total free amine amount was found to be 0.087 mmole/g.

When the test was carried out using beads linked to unprotected SATA or Traut's reagent, the comparable values were 0.021 and 0.18 mmole/g, respectively or 24.1% and 20.7% of the total amine measured by the ninhydrin method. The values from the SATA and Traut's beads are in close agreement and accurately predict the amount of useable amine moieties on the beads.

### EXAMPLE 5

Example 4, supra described how the invention can be used to quantify available or active groups on a solid surface, where the group is an amine group. Additional groups can be quantified the same way. For example, beads which present a tosyl group on their surface were treated with AEDP, (3-[(2-aminoethyl)dithio]propionic acid.HCl), at pH 7. The resulting product can be cleaved by dithiothreitol, TCEP (Tri(2-carboxyethyl)phosphine hydrochloride) or 2-mercaptoethyl-amine leaving a free sulfhydryl group attached to the beads through the usable tosyl. These can then be quantified by reaction with Ellman's reagent as described in Example 4.

AEDP can also be used to quantify the number of usable epoxy groups or carboxyl groups on beads. In the case of the former, the AEDP was added, at pH 7, followed by cleavage as described supra, with DTT, TCEP or 2-mercaptoethanol-amine, leaving a free sulfhydryl group attached to the beads through the usable epoxy group or carboxyl group. These can then be quantified by reaction with Ellman's reagent as described in Example 4.

For carboxyl groups, an additional step was required, i.e., first, 1-ethyl-3-[3-dimethylaminopropyl)carbodiimide hydrochloride was added, at pH8, room temperature, together with the AEDP. The same cleavage step was used.

In each of these cases, following cleavage, Ellman's reagent, as described in example 4, can be used to measure free sulfhydryl groups on the beads. Similarly, the free sulfhydryl groups can be used for further reactions, such as with rapamycin-PMPI.

### EXAMPLE 6

The determination of available or active epoxy, tosyl, and carboxyl groups can also be determined using SACA (S-(2-aminoethyl)ester) in place of AEDP. This provides a protected sulfhydryl group that can be stored or deprotected as was SATA and used. All other criteria are the same.

For solid phases with active carboxyl groups to be determined, the SACA and AEDP molecules may also be used in combination with DCC(N,N¹-dicyclohexyl-carbodiimide).

The foregoing sets forth various aspects of the invention, which include, inter alia a method for determining the active, or usable portion of reactive moieties on a solid phase. As can be seen, supra, the method involves reacting the reactive moieties on the solid phase with a linker. The linker must include a reactive partner for the reactive moiety, and a sulfhydryl group, which is preferably protected. If the sulfhydryl group is protected, following its attachment to the solid phase, the protective element is removed, using art known methods, and the free sulfhydryl group is then reacted with a substance which yields a detectable value, such as a color, a change in pH in the medium, etc., which can be determined. This value is correlatable to the total amount of usable reactive moieties on the solid phase.

If the linker is not protected at the sulfhydryl group, as is the case with, e.g., Traut's reagent, then the reaction with the substance to give a detectable value takes place without the need for deprotecting the sulfhydryl group.

A further aspect of the invention relates to a method for preparing solid phases which have, linked thereto, a first linker molecule containing a sulfhydryl group, as discussed supra, and then a second, linker moiety which reacts with unprotected sulfhydryl groups to bind thereto, and also contains either a ligand of interest, or a substance which binds to a ligand of interest.

Preferably, the second linker contains a maleimido group, but this is not required, as other materials will react with highly reactive sulfhydryl.

The nature of the various components described herein may vary. For example, the solid phase may be a magnetic bead, or any of the materials referred to supra, as well as any solid phase to which reactive moieties may be bound. Literally hundreds of examples of such materials are known to the skilled artisan.

The reactive moiety on the solid phase, as noted, may also vary. An amine group, a tosyl group, a carboxyl group, and an epoxy group are exemplary, but are not inclusive, of the reactive groups which may be attached to the solid phase.

Similarly, the first linker may vary. While SATA is preferred for many applications, it is not the only materials which can be used. AEDP, SACA, and DCC are examples of other materials which provide the requisite sulfhydryl group, preferably in protected form and as noted, Traut's reagent supplies it in unprotected form.

In the embodiment of the invention which relates to determining the usable fraction of reactive moieties, once the sulfhydryl moiety is present on the solid phase, it can be determined with, e.g., Ellman's reagent, or any other material which, upon reaction with a sulfhydryl group, gives a detectable signal.

In the embodiment of the invention where a second linker is attached to the first, compounds such as P-maleimidophenyl isocyanate, DCC, or other molecules which form a stable bond with the sulfhydryl group when unprotected, and which possess a reactive moiety themselves so that they can bind with, e.g., an antibody, such as an FK-506 specific antibody, an immunogen, an antibiotic, an immuno-suppressive drug such as rapamycin, an aptamer or other nucleotide molecule, or any molecule of interest, may be used.

Regardless of the embodiment of the invention, it is important the at the first step of the reaction, and it is preferred that the second step, take place in non-aqueous, or aprotic solvents, as described supra. DMF is exemplified herein, and DMSO is shown in the figures. Additional, but by no means limiting examples of other useful non-aqueous, aprotic solvents are acetonitrile, tetrahydrofuran, acetone, chloroform, methylene chloride, hexane, petroleum ether, ethyl ether, diethyl ether, ethyl acetate, methyl ethyl ketone, benzene, and carbon tetrachloride.

Other features of the invention will be clear to the skilled artisan and need not be elaborated upon herein.

The terms and expression which have been employed are used as terms of description and not of limitation, and there is no intention in the use of such terms and expression of excluding any equivalents of the features shown and described or portions thereof, it being recognized that various modifications are possible within the scope of the invention.

TABLE 1
TOTAL VERSUS USEABLE AMINE ON THE BEAD.

| Source/Method | millimole/g | Amine % |
|---|---|---|
| Manufacturter's Specification | 0.1-0.2 | — |
| Ninhydrin | 0.087 | 100 |
| SATA | 0.021 | 24.1 |
| Traut's Reagent | 0.018 | 20.7 |

1. A method for attaching a ligand to a solid phase comprising:
(i) contacting a reactive group on the surface of said solid phase with a linker molecule which reacts with said reactive group, said linker molecule containing a molecule or sulfhydryl group to attach said linker molecule to said solid phase, said reaction taking place in a non-aqueous, aprotic solvent, and
(ii) reacting said ligand and said linker molecule, wherein said linker comprises a group which reacts with said sulfhydryl group of said linker molecule, to attach said ligand to said solid phase.

2. The method of claim 1, wherein said solid phase is a magnetic bead.

3. The method of claim 1, wherein said ligand comprises an antibiotic.

4. The method of claim 3, wherein said antibiotic is Rapamycin or FK-506.

5. The method of claim 1, wherein said linker molecule is N-succinimidyl-S-acetyl thioacetate (SATA).

6. The method of claim 1, wherein said ligand comprises is p-maleimidophenyl isocyanate.

7. The method of claim 1, wherein said linker molecule is 2-iminothiolane.HCl (Traut's reagent).

8. The method of claim 1, wherein said reactive group is an amine group.

9. The method of claim 1, wherein said reactive group is a tosyl group, a carboxyl group, or an epoxy group.

10. The method of claim 1, wherein said linker is (3-[(2-amino ethyl)dithio]propionic acid.HCl(AEDP), or (S-2-aminoethyl) ester (SACA).

11. A method for determining quantity of usable reactive groups on a solid phase, comprising reacting said solid phase with a linker molecule comprising a moiety which reacts with said reactive group to become affixed thereto, and a sulfhydryl group, and reacting said sulfhydryl group with a substance which yields a detectable signal upon reacting with the sulfhydryl group, as a determination of quantity of usable reactive groups.

12. The method of claim 12, wherein said sulfhydryl group is protected, solid method further comprising deprotecting said sulfhydryl group before reacting said sulfhydryl group with said substance which yields a detectable signal.

13. The method of claim 11, wherein said linker is SATA or Traut's reagent.

14. The method of claim 11, wherein said linker is SACA or AEDP.

15. The method of claim 11, wherein said reactive group is an amine, tosyl, carboxyl, or epoxy group.

16. The method of claim 11, wherein said substance is Ellman's reagent.

17. The method of claim 11, wherein said solid phase is a magnetic bead.

18. The method of claim 1, wherein said non-aqueous, aprotic solvent is DMF, DMSO, chloroform, acetonitrile, tetrahydrofuran, acetine, methylene chloride, hexane, petroleum ether, ethyl ether, ethylacetate, methyl ethyl ketone, benzene, or carbon tetrachloride.

19. The method of claim 1, wherein (ii) is carried out in a non-aqueous, aprotic solvent.
